(19) United States
(12) Patent Application Publication
(10) Pub. No.: US 2007/0010581 A1
(43) Pub. Date: Jan. 11, 2007

(54) Use of 2,5-dihydroxybenzenesulfonic compounds for the treatment of disorders based on an impairment of no production and/or of regulation of edhf function

(76) Inventors: Jose Esteve-Soler, Barcelona (ES); Inigo Saenz De Tejada-Gorman, Madrid (ES); Javier Angulo-Frutos, Valdemoro (ES)

Correspondence Address: PERMAN & GREEN, 425 POST ROAD, FAIRFIELD, CT, 06824, US

(21) Appl. No.: 10/536,780

(22) PCT Filed: Nov. 29, 2003
(86) PCT No.: PCT/FI03/13468

§ 371 Date: Sep. 28, 2006

(30) Foreign Application Priority Data
Nov. 29, 2002 (ES) 200202754

(51) Int. Cl. A61K 31/185

(52) U.S. Cl. 514/553

(57) ABSTRACT

The present invention relates to the use of 2,5-dihydroxybenzenesulfonic compounds for the manufacture of medicament for the regulation of nitric oxide (NO) synthesis and/or the regulation of EDHF (Endothelium-derived-Hyperpolarizing-Factor) in the endothelium of humans or animals, whereby the medicament is administered in a daily dose of the 2,5-dihydroxybenzenesulfonic compounds of formula I of <500 mg.

The present invention relates to the use of 2,5-dihydroxybenzenesulfonic compounds for the manufacture of a medicament for the regulation of nitric oxide (NO) synthesis and/or the regulation of EDHF (Endothelium-Derived-Hyperpolarizing-Factor) in the endothelium of a human or an animal, whereby the medicament is administered in a daily dose of the 2,5-dihydroxybenzenesulfonic compounds of general formula I of <500 mg.

Nitric oxide (NO) exerts critical and diverse functions in the cardiovascular system. Impairment of NO production and/or function plays a major role in a number of cardiovascular disorders and those associated to diabetes or impotency (“Nitric Oxide: A New Paradigm for Second Messengers”, James F. Kerwin Jr. et al., Journal of Medicinal Chemistry, 1995, Volume 38, Number 22, 4343-4362; “Consequences of reduced production of NO on vascular reactivity of porcine coronary arteries after angioplasty importance of EDHF”, Thollon et al., British Journal of Pharmacology, 2002, 136, 1153-1161).

WO97/37647 discloses the use of 2,5-dihydroxybenzenesulfonic compounds for the manufacture of medicaments intended for the normalization of endothelial function, for the treatment of sexual dysfunction, vascular complications of diabetes and for treatment of vascular disorders of endothelial origin. However, according to the prior art, a relatively large daily dosis of one or more of these 2,5-dihydroxybenzenesulfonic compounds, i.e. up to 2000 mg per day, has to be administered to the patient in need of such treatment to obtain the desired beneficial effect.

Administration of a medicament containing 2,5-dihydroxybenzenesulfonic compounds in a large dosis is critical for a number of reasons. Although of rare occurrence, undesired side-effects of these compounds are known, such as gastrointestinal strains, contractions, fever, arthralgia or changes in the blood picture.

Furthermore, many patients have severe problems when confronted with the need to take in a large amount of a medicament.

Thus, the total daily dosis of such a medicament is usually split up into several smaller dosis, which are then administered to the patient several times during the day. However, this requires the patient or, in case of an animal to be treated, the owner of said animal to follow a strict scheme for the intake of the medication, often leading to insufficient compliance.

It was therefore the object of the present invention to provide a medicament for the regulation of nitric oxide (NO) synthesis in the endothelium of humans or animals that avoids the disadvantages of the medicaments known from the prior art. Preferably the medicament should also be useful for the regulation of EDHF (Endothelium-Derived-Hyperpolarizing-Factor), a prime factor in the endothelium-dependent vascular relaxation, as described e.g. in “Human coronary arteriolar dilation to arachidonic acid depends on cytochrome P-450 monooxygenase and Ca²⁺-activated K⁺ channels”, H. Miura, D D. Guterman, Circ. Res., 83, 501-507, 1998; “Endothelium-derived hyperpolarizing factor. Identification and mechanisms of action in human subcutaneous resistance arteries”, Coats et al., Circulation, 103, 1702-1708, 2001; “Characterization of endothelium-derived hyperpolarizing factor in the human forearm microcirculation”, Halcox et al., Am. J. Physiol. Heart Circ. Physiol., 280, H2470-H2477, 2001; “Endothelium-dependent hyperpolarization as a remote anti-atherogenic mechanism”, S. Selemidis, Thomas M. Cocks, TRENDS in Pharmacological Science Vol. 23 No. 5, 213, 2002). Said literature descriptions are incorporated by reference and are part of the disclosure.

Surprisingly, it has now been found that a total daily dose of less than 500 mg of one or more of the 2,5-dihydroxybenzenesulfonic compounds of general formula I given below is sufficient to regulate nitric oxide (NO) synthesis in the endothelium of humans as well as animals.

Moreover, it has been found that the 2,5-dihydroxybenzenesulfonic compounds of general formula I given below are also useful for the regulation of EDHF (Endothelium-Derived-Hyperpolarizing-Factor) when administered in a total daily dose of less than 500 mg to a human or an animal.

Thus, one aspect of the present invention is the use of at least one of the 2,5-dihydroxybenzenesulfonic compounds of the following general formula I,

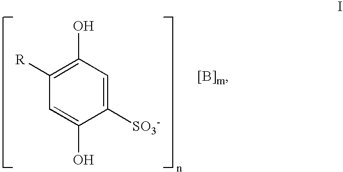

wherein

R represents H or SO₃—,

B represents at least one cation

n represents 1 or 2

m represents 1 or 2,

optionally in form of a pharmaceutically acceptable solvate, for the manufacture of a medicament for the regulation of nitric oxide (NO) synthesis and/or the regulation of EDHF (Endothelium-Derived-Hyperpolarizing-Factor) in the endothelium of a human or an animal, whereby the medicament is administered in a daily dose of the 2,5-dihydroxybenzenesulfonic compounds of general formula I of <500 mg.

The medicament according to the present invention is suitable for the administration to humans as well as animals, whereby the use of at least one of the 2,5-dihydroxybenzenesulfonic compounds of the general formula I for the manufacture of a medicament for the administration to humans is preferred.

The cation B in the 2,5-dihydroxybenzenesulfonic compounds of general formula I may be any physiologically acceptable cation known to those skilled in art, e.g. from P. Heinrich Stahl, Camille G. Wermuth (Editiors), “Handbook of Pharmaceutical Salts—Properties, Selections and Use”, Vedag Helvetica Chimica Acta, Zürich, Switzedand, Wiley-VCH, Weinheim, Germany, 2002, which is hereby incorporated by reference and is part of the disclosure. Those skilled in the art understand that the cation B has to be chosen in such a way that the overall charge of the 2,5-dihydroxybenzenesulfonic compounds of general formula I is neutral.

The present invention encompasses the use of a mixture of at least two of the afore mentioned 2,5-dihydroxybenzenesulfonic compounds of general formula I as well as mixed salts of these compounds, i.e. compounds with different cations B and/or different 2,5-dihydroxybenzenesulfonic residues.

Preferably the cation(s) B of the 2,5-dihydroxybenzenesulfonic compounds of general formula I is (are) selected from the group consisting of Ca²⁺, Mg²⁺, Na⁺, K⁺ and [NH₄₋ₓRₓ]+, wherein x is 0, 1, 2, 3 or 4 and R represents a branched or unbranched C₁₋₄-alkyl-radical. If x is greater than 1, i.e. if two or more alkyl-radicals are present in the [NH₄₋ₓRₓ]+-cation, they may be identical or different, whereby identical alkyl-radicals are preferred.

Preferably the medicament may comprise one or more compounds selected from the group consisting of calcium 2,5-dihydroxybenzenesulfonic (calcium dobesilate), diethylamine 2,5-dihydroxybenzenesulfonic (ethamsylate) and bis(diethylamine)-2,5-dihydroxybenzene-1,4-disulfonate (persilate). Particularly preferably calcium 2,5-dihydroxybenzenesulfonate (calcium dobesilate) is used for the manufacture of the medicament according to the present invention.

The inventively used 2,5-dihydroxybenzenesulfonate compounds of general formula I may also be in the form of solvate, particularly in the form of hydrates. The manufacture of the 2,5-dihydroxybenzenesulfonate compounds of general formula I as well as their solvates may be accomplished by the use of reagents and methods known to those skilled in the art.

The manufacture of calcium 2,5-dihydroxybenzenesulfonate (calcium dobesilate) and diethylamine 2,5-dihydroxybenzenesulfonate (ethamsilate) is known, for example, from “The Merck Index”-13^{th} edtion, Merck & Co., R. Rahway, N.J., USA, 2001. Said literature description is hereby incorporated by reference and is part of the disclosure. The manufacture of bis(diethylamine) 2,5-dihydroxybenzene-1,4-disulfonate (persilate) is known, for example, from French Patent FR 73/17709 (Publication No. 2,201,888). The respective description is hereby incorporated by reference and is part of the disclosure.

The 2,5-dihydroxybenzenesulfonic compounds of general formula I have been found to regulate nitric oxide (NO) synthesis as well as the function of EDHF (Endothelium-Derived-Hyperpolarizing-Factor) in the endothelium of humans and animals in a total daily dose of <500 mg.

The 2,5-dihydroxybenzenesulfonic compounds of general formula I may also be administered to the patients in a lower total daily dose, e.g. 100 to <500 mg, preferably 150 to 450 mg, particularly preferably 200 to 400 mg.

By administering the 2,5-dihydroxybenzenesulfonic compounds of general formula I in a total daily dose of <500 mg the frequency as well as the extent of undesired side effects may be further reduced. The frequency of the administration of the medicament may be reduced to twice per day, preferably once per day, hereby leading to an improvement in patient compliance.

Since the 2,5-dihydroxybenzenesulfonic compounds of general formula I regulate nitric oxide (NO) synthesis as well as EDHF function in the endothelium of humans or animals they are suitable for the preparation of a medicament for the prophylaxis and/or treatment of disorders based on an impairment of nitric oxide (NO) production and/or an impairment of EDHF function (“Calcium Dobesilate: Pharmacology and Future Approaches”, T. Tejerina, E. Ruiz, Gen. Pharmac. Vol. 31, No. 3, 357-360, 1998).

Preferably the afore mentioned 2,5-dihydroxybenzenesulfonic compounds of general formula I may be used or the manufacture of a medicament for the prophylaxis and/or treatment of microcirculation disorders, preferably diabetic retinopathy, sexual dysfunction, particularly erectile dysfunction (“Pharmacological Aspects of Erectile Dysfunction”, John A. Thomas, Jpn. J. Pharmacol. 89, 101-112, 2002), renal disorders, coronary microcirculation disorders and/or peripheral arterial microcirculation disorders.

Depending on the specific embodiment, the medicament of the present invention may also contain, as additional constituents, conventional auxiliary substances known to those skilled in the art.

The medicaments according to the present invention may be produced according to standard procedures known to those skilled in the art, e.g. from the tables of contents from “Pharmaceutics: the Science of Dosage Forms”, Second Edition, Aulton, M. E. (Ed.) Churchill Livingstone, Edinburgh (2002); “Encyclopedia of Pharmaceutical Technology”, Second Edition, Swarbrick, J. and Boylan J. C. (Eds.), Marcel Dekker, Inc. New York (2002); “Modern Pharmaceutics”, Fourth Edition, Banker G. S. and Rhodes C. T. (Eds.) Marcel Dekker, Inc. New York 2002 and “The Theory and Practice of Industrial Pharmacy”, Lachman L., Lieberman H. and Kanig J. (Eds.), Lea & Febiger, Philadelphia (1986). The respective descriptions are incorporated by reference and are part of the disclosure.

In a preferred embodiment of the present invention, the medicament is suitable for oral administration.

If the medicament is suitable for oral administration, it may preferably be in the form of a tablet, a capsule or a suspension.

The medicament of the present invention may also be in the form of multiparticulates, preferably pellets or granules, optionally compressed into a tablet, filled into a capsule or suspended in a suitable liquid. Suitable liquids are known to those skilled in the art.

In a preferred embodiment of the present invention the medicament comprises at least one of the 2,5-dihydroxybenzenesulfonic compounds of general formula I, optionally in form of a solvate, at least partially in a sustained-release form.

By incorporating one or more of the 2,5-dihydroxybenzenesulfonic compounds of general formula I, optionally in form of a solvate, at least partially or completely into a sustained-release form it is possible to extend the duration of their effect, allowing for the beneficial effects of such a sustained release form, e.g. the maintenance of optimal therapeutical plasma or tissue concentrations.

Suitable sustained-release forms as well as materials and methods for their preparation are known to those skilled in the art, e.g. from the tables of contents from “Modified-Release Drug Delivery Technology”, Rathbone, M. J. Hadgraft, J. and Roberts, M. S. (Eds.), Marcel Dekker, Inc., New York (2002); “Handbook of Pharmaceutical Controlled Release Technology”, Wise, D. L. (Ed.), Marcel Dekker, Inc. New York, (2000); “Controlled Drug Delivery”, Vol. I, Basic Concepts, Bruck, S. D. (Ed.), CRC Press Inc., Boca Raton (1983) and from Takada, K. and Yoshikawa, H., “Oral Drug delivery”, Encyclopedia of Controlled Drug Delivery, Mathiowitz, E. (Ed.), John Wiley & Sons, Inc., New York (1999), Vol. 2, 728-742; Fix, J., “Oral drug delivery, small intestine and colon”, Encyclopedia of Controlled Drug Delivery, Mathiowitz, E. (Ed.), John Wiley & Sons, Inc., New York (1999), Vol. 2, 698-728. The respective descriptions are incorporated by reference and are part of the disclosure.

If the medicament according to the present invention comprises at least one of the 2,5-dihydroxybenzenesulfonic compounds of general formula I at least partially in a sustained-release form, said sustained release may preferably be achieved by the application of at least one coating or provision of a matrix comprising at least one sustained-release material.

The sustained-release material is preferably based on an optionally modified, water-insoluble, natural, semisynthetic or synthetic polymer, or a natural, semisynthetic or synthetic wax or fat or fatty alcohol or fatty acid, or on a mixture of at least two of these afore mentioned components.

The water-insoluble polymers used to produce a sustained-release material are preferably based on an acrylic resin, which is preferably selected from the group of poly(meth)acrylates, particularly preferably poly(C₁₋₄)alkyl (meth)acrylates, poly(C₁₋₄)dialkylamino(C₁₋₄)alkyl (meth)acrylates and/or copolymers or mixtures thereof, and very particularly preferably copolymers of ethyl acrylate and methyl methacrylate with a monomer molar ratio of 2:1 (Eudragit NE30D®), copolymers of ethyl acrylate, methyl methacrylate and trimethylammonium ethyl methacrylate-chloride with a monomer molar ratio of 1:2:0.1 (Eudragit RS®), copolymers of ethyl acrylate, methyl methacrylate and trimethylammonium ethyl methacrylate-chloride with a monomer molar ratio of 1:2:0.2 (Eudragit RL®), or a mixture of at least two of the above-mentioned copolymers. These coating materials are commercially available as 30 wt. % aqueous latex dispersions, i.e. as Eudragit RS30D®, Eudragit NE30D® or Eudragit RL30D®, and may also be used as such for coating purposes.

In another embodiment, the sustained-release material is based on water-insoluble cellulose derivatives, preferably alkyl celluloses, particularly preferably ethyl cellulose, or cellulose esters, e.g. cellulose acetate. Aqueous ethyl cellulose dispersions are commercially available, for example, under the trademarks Aquacoat® or Surelease®.

As natural, semisynthetic or synthetic waxes, fats or fatty alcohols, the sustained-release material may be based on carnauba wax, beeswax, glycerol monostearate, glycerol monobehenate, glycerol ditripalmitostearate, microcrystalline wax, cetyl alcohol, cetylstearyl alcohol or a mixture of at least two of these components.

The afore mentioned polymers of the sustained-release material may also comprise a conventional, physiologically acceptable plasticizer in amounts known to those skilled in the art.

Examples of suitable plasticizers are lipophilic diesters of a C₆-C₄₀ aliphatic or aromatic dicarboxylic acid and a C₁-C₈ aliphatic alcohol, e.g. dibutyl phthalate, diethyl phthalate, dibutyl sebacate or diethyl sebacate, hydrophilic or lipophilic citric acid esters, e.g. triethyl citrate, tributyl citrate, acetyltributyl citrate or acetyltriethyl citrate, polyethylene glycols, propylene glycol, glycerol esters, e.g. triacetin, Myvacet® (acetylated mono- and diglycerides, C₂₃H₄₄O₅ to C₂₅H₄₇O₇), medium-chain triglycerides (Miglyol®), oleic acid or mixtures of at least two of said plasticizers.

Aqueous dispersions of Eudragit RS® and optionally Eudragit RL® preferably contain triethyl citrate. The sustained-release material may comprise one or more plasticisers in amounts of, for example, 5 to 50 wt. % based on the amount of polymer(s) used.

The sustained-release material may also contain other conventional auxiliary substances known to those skilled in the art, e.g. lubricants, coloured pigments or surfactants.

The medicament of the present invention may also have at least one enteric coating which dissolves as a function of pH. Because of this coating, the medicament can pass through the stomach undissolved and the compounds of general formula I are only released in the intestinal tract. The enteric coating preferably dissolves at a pH of between 5 and 7.5.

The enteric coating may be based on any enteric material known to those skilled in the art, e.g. on methacrylic acid/methyl methacrylate copolymers with a monomer molar ratio of 1:1 (Eudragit L®), methacrylic acid/methyl methacrylate copolymers with a monomer molar ratio of 1:2 (Eudragit S®), methacrylic acid/ethyl acrylate copolymers with a monomer molar ratio of 1:1 (Eudragit L30D-55®), methacrylic acid/methyl acrylate/methyl methacrylate copolymers with a monomer molar ratio of 7:3:1 (Eudragit FS®), shellac, hydroxypropyl methyl cellulose acetate-succinates, cellulose acetate-phthalates or a mixture of at least two of these components, which can optionally also be used in combination with the above-mentioned water-insoluble poly(meth)acrylates, preferably in combination with Eudragit NE30D® and/or Eudragit RL® and/or Eudragit RS®.

The coatings of the medicament of the present invention may be applied by the conventional processes known to those skilled in the art, e.g. from Johnson, J. L., “Pharmaceutical tablet coating”, Coatings Technology Handbook (Second Edition), Satas, D. and Tracton, A. A. (Eds), Marcel Dekker, Inc. New York, (2001), 863-866; Carstensen, T., “Coating Tablets in Advanced Pharmaceutical Solids”, Swarbrick, J. (Ed.), Marcel Dekker, Inc. New York (2001), 455468; Leopold, C. S., “Coated dosage forms for colon-specific drug delivery”, Pharmaceutical Science & Technology Today, 2(5), 197-204 (1999), Rhodes, C. T. and Porter, S. C., Coatings, in Encyclopedia of Controlled Drug Delivery. Mathiowitz, E. (Ed.), John Wiley & Sons, Inc., New York (1999), Vol. 1, 299-311. The respective descriptions are incorporated by reference and are part of the disclosure.

In another embodiment, the medicament of the present invention contains one or more of the 2,5-dihydroxybenzenesulfonic compounds of general formula I not only in sustained-release form, but also in non-retarded form. By combination with the immediately released form, a high initial dose can be achieved for the rapid onset of the beneficial effect. The slow release from the sustained release form then prevents the beneficial effect from diminishing.

This may be achieved, for example, by a medicament having at least one immediate-release coating comprising at least one of the 2,5-dihydroxybenzenesulfonic compounds of general formula I to provide for rapid onset of the beneficial effect after administration to the patient.

Pharmacological Methods:

The regulation of NO-synthesis by 2,5-dihydroxybenzenesulfonic compounds may be evaluated according to methods known to those skilled in the art, e.g. from “Effects of calcium dobesilate on the synthesis of endothelium-dependent relaxing factors in rabbit isolated aorta”, T. Tejerina et al., British Journal of Pharmacology (1997), 121, 711-716, “In Vitro Effects of Calcium Dobesilate on the Responsiveness of Spontaneously Diabetic Rat Aorta”, T. Tejerina et al., Jpn. J. Pharmacol., 78, 391-394 (1998) and “Dobesilate enhances endothelial nitric oxide syntheses-activity in macro- and microvascular endothelial cells”, Christoph Suschek et al., British Journal of Pharmacology (1997), 122, 1502-1508. The respective literature descriptions are incorporated by reference and are part of the disclosure.

In the following methods for determining the contribution of EDHF (Endothelium-derived hyperpolarizing factor) to the regulation of human penile smooth muscle contractility, for determining the effects of 2,5-dihydroxybenzenesulfonic compounds of general formula I on endothelium-dependent relaxation of penile smooth muscle as well as for determining the effect of these compounds on the erectile responses, in vivo, in a rat model are given.

Vascular Reactivity of Resistance Penile Arteries

Penile small arteries, helicine arteries (lumen diameter 150-400 μm), which are the terminal branches of deep penile arteries, were dissected by carefully removing the adhering trabecular tissue, and arterial ring segments (of 2 mm length) were subsequently mounted on two 40 μm wires on microvascular double Halpern-Mulvany myographs (J.P. Trading, Aarhus, Denmark) for isometric tension recordings. The vascular segments were allowed to equilibrate for 30 min in physiological salt solution (PSS) of the following composition (each given in mM): 119 NaCl, 4.6 KCl; 1.5 CaCl₂, 1.2 MgCl₂, 24.9 NaHCO₃, 11 Glucose, 1,2 KH₂PO₄, 0.027 EDTA in water at 37° C., continuously bubbled with a 95% O₂/5% CO₂ mixture to maintain a pH of 7.4. Passive tension and internal circumference of the vascular segments when relaxed in situ under a transmural pressure of 100 mg Hg (L₁₀₀) were determined. The vascular segments were then set to an internal circumference eqivalent to 90% of L₁₀₀, at which the force development was dose to maximal as described in Mulvany M J, Halpem W., “Contractile properties of small resistance arteries in spontaneously hypertensive and normotensive rats”, Circ. Res., 41, 19-26, 1977. The respective literature description is incorporated by reference and is part of the disclosure. The preparations were then exposed to 125 mM K⁺ (KPSS, equimolar substitution of NaCl for KCl in PSS) and the contractile response was measured. The arteries were contracted with 1 μm norepinephrine (approximately 80% of KPSS induced contraction) and relaxation responses were evaluated by cumulative additions of compounds to the chambers. The arterial segments considered as lacking functional endothelium did not relax to 10 μM acetylcholine.

Organ Chamber Studies

Strips of corpus cavernous tissue (3×3×7 mm) were immersed in 8 ml organ chambers containing PSS, maintained at 37° C. and aerated with 95% O₂/5% CO₂ mixture to maintain a pH of 7.4. Each tissue strip was incrementally stretched to optimal isometric tension, as determined by maximal contractile response to 1 μM phenylephrine, as described in Saenz de Tejada et al., “A Nitric Oxidelike factor mediates nonadrenergenic-noncholinergic neurogenic relaxation of penile corpus cavemosum smooth muscle”, J. Clin. Invest. 88, 112-118. The respective literature description is incorporated by reference and is part of the disclosure. Tissues were contracted with 0.5-3 μm phenylephrine (80% of KPSS induced contraction and relaxation responses were evaluated by cumulative additions of compounds to the chambers.

Erectile Responses to Cavernosal Nerve Stimulation in Anesthetized Rats.

Male Sprague-Dawley rats were anesthetized with ketamine (60 mg/kg). The surgical procedure consisted of dissection and isolation of the right cavernous nerve through an abdominal midline incision and exposure of penile crura through a transverse perineal incision. Intracavemosal pressure (ICP) measurements were accomplished by insertion into the right crus of a 23-gauge needle connected to a disposable pressure transducer (Abbott, Sligo, Ireland) and a data acquisition system (ADInstruments, Castle Hill, Australia). Left carotid artery and right external jugular vein were catheterized for constant blood pressure measurement and saline or drug infusion, respectively. Electrical stimulation was applied by a delicate platinum bipolar hook electrode connected to a stimulator and current amplifier (Cibertec, Madrid, Spain). Parameters of electrical stimulation consisted of pulses with a duration of 1 ms and 1.5 mA of current intensity for 1 minute. Frequency-response curves were performed by applying stimulation at 1, 3 and 10 Hz at 3 minute intervals.

For evaluation of acute effects of a 2,5-dihydroxybenzenesulfonic compound of general formula I on erectile responses, a control stimulation at 1, 3 and 10 Hz was performed and, after an stabilization period, the respective compound (10 mg/kg) dissolved in 20% hydroxy-propyl-_{β}-cyclodextrin (HP_{β}CD) or the vehicle alone were intravenously administered. The stimulation was repeated at 60 min after the administration of the respective compound or vehicle.

The present invention is illustrated below with the aid of examples. These illustrations are given solely by way of example and do not limit the general spirit of the present invention.

### EXAMPLES

### Example 1

Hard Gelatin Capsule comprising calcium dobesilate:

| | |
|---|---|
| Calcium dobesilate | 0.400 g |
| Cellulose | 0.023 g |
| Magnesiumstearate | 0.007 g |
| Colloidal silicon dioxide | 0.005 g |
| Total weight | 0.435 g |

Calcium dobesilate, Cellulose, Magnesiumstearate and Colloidal silicon dioxide in the afore mentioned amounts were thoroughly mixed in a conventional mixer and then filled into a conventional hard gelatin capsule.

### Example 2

Tablet comprising calcium dobesilate:

| | |
|---|---|
| Calcium dobesilate | 0.2000 g |
| Maize starch | 0.0650 g |
| Lactose | 0.0520 g |
| Povidone K-30 | 0.0175 g |
| Citric acid monohydrate | 0.0125 g |
| Magnesiumstearate | 0.0020 g |
| Sodium bisulfite | 0.0010 g |
| Total weight | 0.3500 g |

Calcium dobesilate, Maize starch, Lactose, Povidbne K-30, Citric acid monohydrate, Magnesiumstearate and Sodium bisulfite in the afore mentioned amounts were thoroughly mixed in a conventional mixer and then compressed into a tablet on a conventional tabletting press.

Pharmacological Methods:

Drugs and Materials:

Phenylephrine, norepinephrine (arterenol), acetylcholine, indomethacin, N^{G}-nitro-L-arginine (L-NNA), apamin, charybdotoxin and hydroxy-propyl-_{β}-cyclodextrin (HP_{β}CD) were obtained from Sigma Chemical Co. (St. Louis, Mo., USA). Miconazole was obtained by RBI (Natick, Mass., USA). Calcium dobesilate (calcium dihydroxy-2,5 benzenesulfonate, Doxium®) was obtained from Dr. Esteve Laboratories (Barcelona, Spain).

Data Analysis:

Relaxation responses are expressed as percentage of total relaxation (loss in tone) induced by the addition of 0.1 mM papaverine HCl to the chambers at the end of the experiment. All data are expressed as mean±standard error. Complete concentration-response or frequency-response curves were obtained and compared by a two-factor analysis of variance (ANOVA) statistical test using StatView sotfware for Apple computers. Erectile responses were determined by measuring the area under the curve (AUC) of the intracavemosal pressure increases to rat cavernosal nerve stimulation normalized by mean arterial pressure values. The complete frequency-response curves were compared by a two-factor ANOVA test.

Role of EDHF in Endothelium-Dependent Relaxation of Human Corpus Cavernosum Trabecular Strips and Human Penile Resistance Arteries (HPRA):

In strips of trabecular tissue, acetylcholine (ACh; 1 nM to 10 μM) produced concentration-dependent relaxation which was nearly abolished after combined treatment with the NO synthase (NOS) inhibitor, N^{G}-nitro-L-arginine (L-NNA; 100 μM) and the cyclooxygenase (COX) inhibitor, indomethacin (5 μM). Conversely, in penile arteries although the treatment with L-NNA (100 μM) and indomethacin (5 μM) significantly reduced ACh-induced relaxation, an important component of relaxation of these arteries remained. The ACh-induced relaxations in the presence of NOS and COX inhibition, were abolished by contrasting the HPRA with a high extracellular K⁺ concentration (35 mM) or by treating the arteries with a combination of the Ca²⁺-dependent K+-channel blockers, apamin (APA; 100 nM) and charybdotoxin (CTX; 100 nM).

Effects of Calcium Dobesilate on Endothelium-Dependent Relaxation of Human Penile Smooth Muscle:

Calcium dobesilate (10 μM) markedly potentiated ACh-induced relaxation of HPRA. This potentiation by calcium dobesilate (10 μM) was not significantly affected by the combined inhibition of NOS and COX, but was abolished if the arteries were exposed to a high potassium concentration (35 mM).

Exposure of HPRA to a combination of APA (100 nM) and CTX (100 nM) that blocked ACh induced relaxation resistant to NOS and COX inhibition, abolished the potentiating effects of calcium dobesilate. Finally, miconazole (0.3 mM) significantly reduced the potentiating effects of calcium dobesilate of ACh-induced relaxation of HPRA resistant to NOS and COX inhibition.

Effects of Calcium Dobesilate on Erectile Responses in Anesthetized Rats:

Electrical stimulation of the cavernosal nerve in anesthetized rats produced frequency-dependent intracavemosal pressure (ICP) increases which were not modified by the treatment with vehicle (20% HP_{β}CD). Intravenous administration of calcium dobesilate (10 mg/kg) significantly enhanced the erectile responses to cavernosal nerve stimulation.

These afore mentioned results show that EDHF participates in the endothelium-dependent relaxation of human penile resistance arteries. The regulation of EDHF by calciumdobesilate is shown.

The concentration of calcium dobesilate used in the in vitro experiments described above is in the range of plasma levels achieved after an oral dose of <500 mg. Even at this small dose calcium dobesilate results in enhanced erectile responses.

1-25. (canceled)

26. Use of at least one of the 2,5-dihydroxybenzenesulfonic compounds of general formula I, 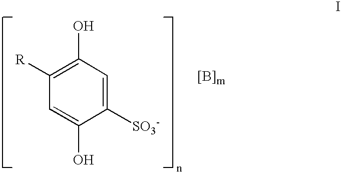
wherein
R represents H or SO₃—,
B represents at least one cation
n represents 1 or 2
m represents 1 or 2,
optionally in form of a pharmaceutically acceptable solvate, for the manufacture of a medicament for the prophylaxis and/or treatment of sexual dysfunction in humans, whereby the medicament is administered in a daily dose of the afore mentioned compounds of formula I of <500 mg.

27. Use according to claim 26, characterised in that the cation(s) B is (are) selected from the group consisting of Ca²⁺, Mg²⁺, Na⁺, K⁺ and [NH₄₋ₓRₓ]⁺, whereby x is 0, 1, 2, 3, or 4 and R represents a branched or unbranched C₁₋₄-alkyl-radical that may be the same or different for x>1.

28. Use according to claim 26, characterized in that the compound of general formula I is calcium 2,5-dihydroxybenzenesulfonate (calcium-dobesilate).

29. Use according to claim 26, characterized in that the compound of general formula I is diethylamine 2,5-dihydroxybenzenesulfonate (ethamsylate).

30. Use according to claim 26, characterized in that the compound of general formula I is bis(diethylamine)-2,5-dihydroxybenzene-1,4-disulfonate (persilate).

31. Use according to claim. 26, characterized in that medicament is administered in a daily dose of compounds of general formula I of 100 to <500 mg, preferably 150 to 450 mg, particularly preferably 200 to 400 mg.

32. Use according to claim 26 for the prophylaxis and/or treatment of erectile dysfunction.

33. Use according to claim 26, characterized in that the medicament is suitable for oral administration.

34. Use according to claim 33, characterized in that the medicament is in the form of a tablet, a capsule or a suspension.

35. Use according to claim 33, characterized in that the medicament is in form of multiparticulates, preferably pellets or granules, optionally compressed into a tablet, filled into a capsule or suspended in a suitable liquid.

36. Use according to claim 26, characterized in that the medicament comprises at least one of the compounds of general formula I at least partially in a sustained-release form.

37. Use according to claim 36, characterized in that the medicament has at least one coating or matrix comprising at least one sustained-release material.

38. Use according to claim 37, characterized in that the sustained-release material is based on an optionally modified, water-insoluble, natural, semisynthetic or synthetic polymer, or a natural, semisynthetic or synthetic wax or fat or fatty alcohol or fatty acid, or on a mixture of at least two of these afore mentioned components.

39. Use according to claim 38, characterized in that the water-insoluble polymer is based on an acrylic resin, which is preferably selected from the group of poly(meth)acrylates, poly(C₁₋₄)dialkylamino(C₁₋₄)alkyl (meth) acrylates and/or copolymers thereof or a mixture of at least two of the afore-mentioned polymers.

40. Use according to claim 38, characterized in that the water-insoluble polymers are cellulose derivatives, preferably alkyl cellulose and particularly preferably ethyl cellulose, or cellulose esters.

41. Use according to claim 38, characterized in that the wax is carnauba wax, beeswax, glycerol monostearate, glycerol monobehenate, glycerol ditripalmitostearate, microcrystalline wax or a mixture of at least two of these components.

42. Use according to claim 38, characterized in that the polymers have been used in combination with one or more plasticizers.

43. Use according to claim 32, characterized in that the medicament comprises an enteric coating.

44. Use according to claim 26, characterized in that the medicament comprises at least one immediate-release coating comprising at least one of the compounds of general formula I.
